(19) United States
(12) Patent Application Publication
(10) Pub. No.: US 2003/0157566 A1
(43) Pub. Date: Aug. 21, 2003

(54) Arrays of n aqueous polymer dispersions and m formulations of aqueous polymer dispersions

(76) Inventors: Sebastian Koltzenburg, Waldsee (DE); Dirk Wulff, Schifferstadt (DE); Harald Rockel, Neustadt (DE); Wolfgang Schrof, Neuleiningen (DE); Peter Rossmanith, Mannheim (DE); Walter Machtle, Ludwigshafen (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/333,065

(22) PCT Filed: Jul. 11, 2001
(86) PCT No.: PCT/EP01/08007

(30) Foreign Application Priority Data
Jul. 18, 2000 (DE) 100 34 893.9

(51) Int. Cl. G01N 33/53; C08F 2/00; G01N 33/543

(52) U.S. Cl. 435/7.1; 436/518; 526/72

(57) ABSTRACT

The present invention relates to combinatorial processes for preparing an array of n aqueous polymer dispersions by emulsion polymerization and for preparing an array of m formulations of polymer dispersions, and also to the arrays themselves.

The processes are conducted at n or m spatially separate locations for the n or m mixtures, respectively. To conduct the emulsion polymerization, monomers, initiator, dispersants and, if desired, further components are reacted with one another in water as the reaction medium. To prepare the formulations, aqueous polymer dispersions are mixed with other aqueous polymer dispersions and/or further components. With all processes, each of the n or m mixtures differs from every other mixture in at least one parameter in each case which may have an influence on the properties of the resultant polymer dispersions or formulations, respectively.

The advantage of the invention is that by means of these processes it is possible rapidly to prepare and characterize a large number of aqueous polymer dispersions and formulations. This leads to an acceleration in the development process for the preparation of aqueous polymer dispersions and formulations having optimized properties.

The invention relates to a process for preparing an array of n aqueous polymer dispersions, to a process for preparing an array of m formulations of aqueous polymer dispersions, and to arrays preparable accordingly.

A development in the production and investigation of new chemical compounds, alongside classical chemistry, which is aimed at the synthesis and investigation of individual substances, is that of what is known as combinatorial chemistry. The aim of the latter is to provide a large number of substances for, say, pharmacological tests, in as short a time as possible. Systematic variations in molecules are made possible by using reagents and building blocks repeatedly but in each case with different linkages. In order to produce a basic structure, also referred to as a template, a plurality of very different substituents are varied systematically. In this way, a substance library—an array—is obtained which contains a large number of compounds and is diversified by way of the variety of the structure clusters.

In the mix and split process, a large number of reactants are reacted stepwise with a plurality of reagents, in each case in a one-pot reaction, and the resulting reaction mixture is then investigated as to whether it exhibits the desired properties, e.g., a pharmacological activity. If an activity is found for such a reaction mixture, it is necessary in a further step to determine which specific substance in the reaction mixture is responsible for the activity. In addition to the great complexity of determining the compound which is actually active, it is difficult, furthermore, with a large number of reactants, to rule out unwanted secondary reactions and also phantom effects in biological test systems caused by the interaction of two or more compounds.

In another approach of combinatorial synthesis, a large number of individual compounds are synthesized by controlled metering and reaction of a series of reactants in a large number of different reaction vessels. This process can be carried out in parallel and automated. Preferably, after reaction has taken place, there is one reaction product in each individual reaction vessel, so that it a substance is found to have pharmacological activity the starting materials used to prepare it are immediately known. In order to be able to exploit the time advantage of a combinatorial synthesis to the optimum, it is absolutely necessary here to develop rapid analytical methods which as far as possible can be parallelized and automated.

In addition to first applications of this more specific combinatorial synthesis in the course of the search for new pharmacologically active substances and in peptide synthesis, the synthesis process has more recently been extended to embrace low molecular mass organic compounds, organic and inorganic catalysts, and polymers, as well.

The field of polymers also includes aqueous polymer dispersions. Aqueous polymer dispersions are fluid systems comprising polymer particles in the form of polymer coils as the disperse phase in the aqueous dispersion medium, these particles or coils being present in substantially stable disperse distribution. The diameter of the polymer particles is generally predominantly in the range from 0.01 to 5 μm, in many cases permanently in the range from 0.01 to 1 μm. The stability of the disperse distribution often extends over a period of ≧1 month, in many cases even over a period of ≧3 months. Based on the overall volume of the aqueous polymer dispersion, its polymer volume fraction is normally from 10 to 70% by volume.

Like polymer solutions when the solvent has evaporated, aqueous polymer dispersions, when the aqueous dispersion medium is evaporated, have the ability to form polymer films, which is why aqueous polymer dispersions are employed diversely as binders, for paints or compositions for the coating of wood, roofs or leather, and as pressure sensitive adhesives, for example.

Thus aqueous polymer dispersions are used for coating and adhesive bonding, and also for sealing and impregnating. Preference is given to their use as pressure sensitive adhesives featuring enhanced cohesion and as binders for moldings or finely divided coating compositions comprising mineral and/or organic substances such as fillers and pigments.

U.S. Pat. No. 5,985,356 deals very generally with methods and apparatus for the preparation of arrays on the surface of a substrate, which is divided into n different reaction sites for n reaction mixtures. The possibility of the preparation of arrays of nonbiological, organic polymers is mentioned. There is detailed description of the free radical copolymerization of styrene with acrylonitrile in toluene at 60° C. The polymers thus obtained may be investigated, for example, in respect of their hardness. A disadvantage of said methods is that it is impossible to rule out fully contamination by substance mixtures from adjacent synthesis locations.

WO 99/52962 describes a method for the parallel synthesis of a substance library of copolymers, starting from at least two basic structures of structurally different monomers. Described at length is the synthesis of copolymers by polycondensation of dicarboxylic acids with diphenols. The polymerizations are conducted both in suspension and at the phase boundary or in bulk, in the presence or absence of catalysts. Besides polycondensation reactions, free radical polymerization reactions and ionic polymerization reactions are also employed.

WO 99/51980 describes different methods and apparatus for the rapid characterization of polymer libraries. Proposed techniques for doing so include liquid chromatography techniques such as gel permeation chromatography (GPC), which are suitable for investigating liquid polymer samples such as polymer solutions, suspensions and emulsions. The polymers are characterized using, in particular, the weight average of the molar mass, M_{w}, and the number average of the molar mass, Mₙ, the molar mass distribution and its width, expressed through the ratio M_{w}/Mₙ, and also the polydispersity and the hydrodynamic radius. The investigations are carried out both in parallel and in series. In some cases, parallel and serial investigation methods are combined with one another. Preference is given to the investigation of nonbiological polymers. Exemplary applications specified include combinatorial polymer research and industrial process control.

To date, combinatorial synthesis has not been applied to polymers prepared by the emulsion polymerization process.

It is an object of the present invention to develop a methodology for rapid preparation of a substance library from aqueous polymer dispersions in order thus to enable polymer properties to be optimized more rapidly than by conventional synthesis processes.

We have found that this object is achieved by a process for preparing an array of n polymer dispersions by emulsion polymerization in n parallel reaction mixtures, comprising the steps of

(a) providing two or more monomers, one or more initiators, one or more dispersants, water as reaction medium and, if desired, further useful components suitable for emulsion polymerization at n spatially separate reaction sites for the n reaction mixtures,

(b) causing the simultaneous or successive reaction of the two or more monomers and, if appropriate, of the further useful components, each of the n reaction mixtures differing from any other reaction mixture in at least one reaction parameter in each case, selected from the group consisting of nature and amount of the monomers, dispersants, initiators and further suitable components used, amount of the reaction medium, reaction temperature, reaction pressure, type of mixing, mode of conduct of the polymerization reaction, and other parameters which may have an influence on the properties of the resulting polymer dispersions,

(c) carrying out parallel and/or serial characterization of the resulting n polymer dispersions for at least one property which is of interest,

n being an integer greater than or equal to 1.

The advantage of the invention is that, by virtue of the process of the invention, it is possible to prepare and characterize a large number of aqueous polymer dispersions. This leads to an acceleration of the development process. Moreover, statements concerning structure/property relationships are possible by virtue of the large number of data obtained as a result. Similarly, it is possible to investigate more rapidly the influence of reaction parameters such as temperature and pressure.

The term emulsion polymerization embraces both conventional emulsion polymerization and miniemulsion polymerization. The size of the particles present in the emulsion in the case of conventional emulsion polymerization and miniemulsion polymerization is approximately the same. The particles typically have a diameter of from 200 to 900 nm. In the case of a miniemulsion polymerization, polymerization takes place in monomer droplets which have already been preformed, and which are merely polymerized to completion. This process is advantageous in connection with the addition of monomers of extremely low water solubility, or other noncopolymerizable, oil-soluble compounds which remain enclosed in the particles.

The provision of two or more monomers, one or more initiators, one or more dispersants, water as reaction medium, and further components comprises the manual or automatic transport of the abovementioned components to the n reaction sites. In one preferred embodiment of the invention, a metering robot is used. The reactants may be present in gaseous, solid or liquid form or dispersed in solution. Preference is given to a continuous feed of components, which feed may be linear or nonlinear.

The nature, number and amount of the monomers used is dependent on the desired composition of the aqueous polymer dispersions, and their simultaneous or successive provision on the nature of the reaction regime. In general, the monomers may be added either as they are or as emulsions. Addition as emulsions is preferred; i.e., the monomers are emulsified in water with dispersant prior to the addition. Normally, the dispersants are provided in dilute aqueous form. Before being used, however, they are usually diluted further with water. It is also possible to use mixtures of dispersants.

In general, the monomers are emulsified in water individually or in a mixture with dispersant. The initiator or initiators is or are provided as a solution in water or emulsified in water with dispersant.

Monomers

In general, at least two monomers are reacted with one another. In one preferred embodiment of the invention, three monomers are reacted with one another: a monomer containing at least one monoethylenically unsaturated group, a monomer whose solubility in water is greater than that of the first, and a monomer which increases the internal strength of the films formed from the aqueous polymer dispersions.

Suitable monomers containing at least one monoethylenically unsaturated group for the process of the invention are free radically polymerizable monomers such as olefins, e.g., ethylene, vinylaromatic monomers such as styrene, α-methylstyrene, α-chlorostyrene or vinyltoluenes, esters of vinyl alcohol and monocarboxylic acids having 1 to 18 carbon atoms, such as vinyl acetate, vinyl propionate, vinyl n-butyrate, vinyl laurate and vinyl stearate, esters of α,β-monoethylenically unsaturated monocarboxylic and dicarboxylic acids having preferably 3 to 6 carbon atoms, such as acrylic acid, methacrylic acid, maleic acid, fumaric acid and itaconic acid, with alkanols having generally 1 to 12, preferably 1 to 8 and with particular preference 1 to 4 carbon atoms, such as methyl, ethyl, n-butyl, isobutyl and 2-ethylhexyl acrylate and methacrylate, dimethyl maleate or n-butyl maleate, nitriles of α,β-monoethylenically unsaturated carboxylic acids, such as acrylonitrile, and also conjugated C₄-C8 dienes such as 1,3-butadiene and isoprene. The abovementioned monomers are generally the principal monomers, which, based on the total amount of monomers to be polymerized by the process of free radical aqueous emulsion polymerization, normally account for a proportion of more than 50% by weight. As a general rule, these monomers are only of moderate to low solubility in water under standard conditions (25° C., 1 atm).

Monomers which have an increased solubility in water under the above-mentioned conditions are, for example, α,β-ethylenically unsaturated monocarboxylic and dicarboxylic acids and their amides, such as acrylic acid, methacrylic acid, maleic acid, fumaric acid, itaconic acid, acrylamide and methacrylamide, and also vinylsulfonic acid and its water-soluble salts, and N-vinylpyrrolidone.

The abovementioned monomers having an increased solubility in water are normally copolymerized only as modifying monomers in amounts, based on the overall amount of the monomers to be polymerized, of less than 50% by weight, generally from 0.5 to 20, preferably from 1 to 10% by weight.

Monomers which normally increase the internal strength of the films formed from the aqueous polymer dispersions normally contain at least one epoxy, hydroxyl, N-methylol, N-hydroxymethylamino, N-hydroxymethylaminocarbonyl or carbonyl group, or at least two nonconjugated ethylenically unsaturated double bonds. Examples are N-alkylolamides of α,β-monoethylenically unsaturated carboxylic acids having 3 to 10 carbon atoms, esters of α,β-monoethylenically unsaturated carboxylic acids, having 3 to 10 carbon atoms, with alkenols having 1 to 4 carbon atoms, among which N-methylolacrylamide and N-methylolmethacrylamide are especially preferred, and monomers containing two alkenyl—such as vinyl or methylene—radicals. Particularly advantageous in this context are the diesters of dihydric alcohols with α,β-monoethylenically unsaturated monocarboxylic acids, among which acrylic acid and methacrylic acid are preferred. Examples of such monomers containing two nonconjugated ethylenically unsaturated double bonds are alkylene glycol diacrylates and dimethacrylates such as ethylene glycol diacrylate, 1,3-butylene glycol diacrylate, 1,4-butylene glycol diacrylate, and propylene glycol acrylate, divinylbenzene, vinyl methacrylate, vinyl acrylate, allyl methacrylate, allyl acrylate, diallyl maleate, diallyl fumarate, methylenebisacrylamide, cyclopentadienyl acrylate or triallyl cyanurate. Also of particular importance in this context are the C₁-C₈ hydroxyalkyl esters of methacrylic and acrylic acid, such as n-hydroxyethyl, n-hydroxypropyl or n-hydroxybutyl acrylate and methacrylate, and also compounds such as diacetoneacrylamide and acetylacetoxyethyl acrylate and methacrylate. The abovementioned monomers are usually copolymerized in amounts of from 0.5 to 10% by weight, based on the overall amount of the monomers to be polymerized.

Initiators

Suitable initiators are all those initiators which are able to trigger a free radical aqueous emulsion polymerization, such as peroxides and azo compounds. Redox initiator systems may also be used. Preference is given to the use of peroxodisulfuric acid and/or its alkali metal salts and/or its ammonium salt. It is also possible to use mixtures of initiators. Preferably, the amount of the free radical initiator systems used, based on the overall amount of the monomers to be polymerized, is from 0.1 to 2% by weight.

Dispersants

In the context of free radical aqueous emulsion polymerization it is common also to use dispersants, which ensure the stability of the aqueous polymer dispersion produced. Suitable such dispersants include both the protective colloids commonly used to conduct free radical aqueous emulsion polymerizations, and also emulsifiers, or mixtures of protective colloids and/or emulsifiers.

Examples of suitable protective colloids are polyvinyl alcohols, cellulose derivatives or vinylpyrrolidone copolymers. A detailed description of further suitable protective colloids is given in Houben-Weyl, Methoden der Organischen Chemie, Volume XIV/1, Makromolekulare Stoffe [Macromolecular substances], Georg-Thieme-Verlag, Stuttgart, 1961, pp. 411-420. As dispersants it is preferred to use exclusively emulsifiers, whose relative molecular weights, unlike those of the protective colloids, are usually below 1000. They may be anionic, cationic or nonionic in nature. Where mixtures of surface-active substances are used, the individual components must be compatible with one another, something which in case of doubt can be checked on the basis of a few preliminary tests. In general, anionic and cationic emulsifiers are in each case compatible with one another and with nonionic emulsifiers. In contrast, anionic and cationic emulsifiers are generally incompatible with one another. Customary emulsifiers are, for example

ethoxylated mono-, di- and trialkylphenols having an ethylene oxide degree (EO degree) of from 3 to 50% by weight and alkyl radicals with 4 to 9 carbon atoms,

ethoxylated fatty alcohols having an EO degree of from 3 to 50% by weight and alkyl radicals with 8 to 36 carbon atoms,

alkali metal salts and ammonium salts of alkyl sulfates having alkyl radicals with 8 to 12 carbon atoms,

alkali metal salts and ammonium salts of sulfuric monoesters of ethoxylated alkanols having an EO degree of from 4 to 30% by weight and alkyl radicals of 12 to 18 carbon atoms,

alkali metal salts and ammonium salts of sulfuric monoesters of ethoxylated alkylphenols having an EO degree of from 3 to 50% by weight and alkyl radicals with 4 to 9 carbon atoms,

alkali metal salts and ammonium salts of alkylsulfonic acids having alkyl radicals with 12 to 18 carbon atoms, and

alkali metal salts and ammonium salts of alkylarylsulfonic acids having alkyl radicals with 9 to 18 carbon atoms.

Further suitable emulsifiers are given in Houben-Weyl, Methoden der Organischen Chemie, Volume XIV/1, Makromolekulare Stoffe, Georg-Thieme-Verlag, Stuttgart, 1961, pp. 192-208.

Moreover, compounds of the formula (I) have also been found to be suitable surface-active substances

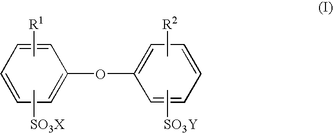

in which R¹ and R² are hydrogen or C₄ to C₂₄ alkyl but are not both hydrogen, and X and Y may be alkali metal ions and/or ammonium ions. Preferably, the compounds (I) are used per se in the process of the invention, and with particular preference they are used in a mixture with ethoxylated fatty alcohols having an EO degree of from 3 to 50% by weight and alkyl radicals with 8 to 36 carbon atoms, as dispersants. The compounds (I) are described in U.S. Pat. No. 4,269,749 and are available commercially.

In general, the amount of dispersant used is from 1 to 3% by weight, based on the monomers for free radical polymerization.

The abovementioned dispersants are suitable very generally for stabilizing the direct process products of the invention. The direct process products of the invention also, however, include aqueous polymer dispersions of self-emulsifying addition polymers, i.e., of addition polymers which have ionic groups, which have the capacity to bring about stabilization on the basis of the repulsion of like charges. Preferably, the direct process products of the invention exhibit anionic stabilization (in particular, anionic dispersants).

Further Useful Components

Further useful components suitable for the polymerization process employed include, for example

regulators by means of which the molar mass is reduced,

dyes,

seed particles, as described in EP-B2 129 699 and EP-B1 22 633, and

other oil-soluble components which are not copolymerized.

Examples of regulators used are compounds containing a thiol group, such as tert-butyl mercaptan, 2-ethylhexyl thioglycolate, mercaptoethanol, mercaptopropyltrimethoxysilane, and tert-dodecyl mercaptan. The proportion of the regulators is preferably from 0 to 0.3% by weight, with particular preference from 0.02 to 0.3% by weight, based on the polymer.

In accordance with the invention, the oil-soluble dyes include organic optical brighteners, i.e., organic molecules having an extended, conjugated π electron system, which unlike the π electron system of conventional organic dyes absorbs ultraviolet radiation rather than radiation in the visible region and emits this UV radiation again as a bluish fluorescence. Optical brighteners increase the whiteness, for example, of white substrates treated with them, such as paper or fabrics.

Seed particles used comprise small polymer beads; for example, 0.1% by weight of linear polystyrene beads. This makes it possible to control particle formation, as described in EP-B 40 419 and Encyclopedia of Polymer Science and Technology, John Wiley & Sons Inc., New York, 1966, Vol. 5, p. 847. When very small polymer beads with a particle diameter of from 20 to 30 nm are used as nuclei, they are referred to as fine seed particles.

Examples of noncopolymerizable, oil-soluble compounds are poly-n-butyl acrylates (e.g., Acronal® A 150 F from BASF AG), high temperature solution polymers of n-butyl acrylate (e.g., PnBa), resins such as rosins, which are described in Ullmanns Encycl. Techn. Chem. 1976, 4th edition, Vol. 12, pp. 525-538, and hydrocarbon resins (e.g., Kristalex F 85 from Hercules), which are described in Encycl. Polym. Sci. Eng. 1987, Vol. 7, pp. 758-782. Also suitable are glycerol esters of highly hydrogenated rosins (e.g., Floral® 85 E from Hercules) and polystyrenes, which are described in C. M. Miller et al., J. Polym. Sci.: Part A: Polym. Chem. 1994, 32, pp. 2365-2376. However, it is also possible to use other water-insoluble, oil-soluble substances such as aliphatic and aromatic hydrocarbons (e.g., hexadecane), film forming auxiliaries or plasticizers such as mixtures of di-n-butyl esters of C₄ to C₆ dicarboxylic acids (e.g., Plastilit® 3060 from BASF AG).

Reaction Sites

Reaction sites chosen may be all kinds of reaction vessels made of metal, such as aluminum or stainless steel, or of polymers, such as Teflon or polyethylene, or of glass, which are suitable for the conduct of an emulsion polymerization. The reactions may be conducted, for example, in the wells of a microtitre plate (see WO 99/51980) or in glass vessels.

In one preferred embodiment of the invention, use is made of an automated, parallelized and miniaturized synthesis system as described in WO 00/09255. This synthesis system allows more than 20, preferably more than 100, with particular preference more than 1 000, with very particular preference more than 10 000, parallel automated reactions to be carried out in reactor blocks. Reaction volumes are from 0.01 μl to 100 ml, preferably from 1 μl to 100 ml, with particular preference from 0.1 ml to 100 ml, with very particular preference from 1 ml to 100 ml.

It is possible advantageously to use jacketed vessels whose jacket can be charged with a heating medium for the purpose of controlling the reaction temperature. The temperature of the heating medium may be controlled by means of a thermostat. Cooling fingers made in the reactor can be used to suppress the evaporation of volatile components, so ensuring the reproducibility of the experiments.

Reaction Procedure

The reaction may be carried out in a variety of ways. How it is carried out is determined by the desired nature and composition of the products.

The two or more monomers, the initiator or initiators, the dispersant or dispersants and any further useful components suitable for the polymerization process used may be reacted with one another simultaneously or in succession in the aqueous reaction medium. The reaction may be carried out in one or more stages. The monomers, initiators and dispersants, which may be identical or different, may be added either simultaneously or in succession. The reactants may be added continuously, semicontinuously or batchwise, continuous addition being preferred. In the case of batchwise addition, the individual reactants are each added in one go. Continuously, the individual reactant may be added both with a constant feed rate and with a graduated feed rate. The point in time from which the reaction mixture is brought to the reaction temperature is likewise dependent on the nature of the reaction regime. Other kinds of reaction regime are also conceivable, known to the skilled worker, and fall within the sphere of validity of this invention.

If desired, further useful components suitable for the polymerization process, such as seed particles, dyes or oil-soluble particles, may be present both in the initial charge and in the feed.

In one embodiment of the invention, all monomers, emulsified in water with dispersant, are introduced as the initial charge and the initiator with dispersant in water is added as a feed.

In another embodiment of the invention, the initiator with dispersant in water is introduced as the initial charge and the two or more monomers, emulsified in water with dispersant, are added as a feed. The two or more monomers are added either individually in succession or as a monomer mixture.

In a further embodiment of the invention, a portion of the monomer with dispersant in water is introduced as the initial charge and the initiator emulsified in water with the remaining monomers, with dispersant, is added as a feed.

In a preferred embodiment of the invention, generally from 0 to 50, preferably from 0 to 30, with particular preference from 0 to 20, and especially advantageously from 0 to 10% by weight of the overall amount of the monomers are introduced in water with initiator and dispersant and this initial charge is brought to the desired reaction temperature. The predominant amount of the monomers to be polymerized, generally from 50 to 100, preferably from 70 to 100, with particular preference from 80 to 100, and especially advantageously from 90 to 100% by weight of their total amount, are added to the polymerization vessel only after the beginning of the free radical aqueous emulsion polymerization, in accordance with the progress of the polymerization of the monomers already present in the polymerization vessel. In general, the addition is made by continuous supply (generally as a straight monomer feed or preemulsified in aqueous phase) in such a way that at least 80, preferably at least 90 and with very particular preference at least 95% by weight of the monomers already present in the polymerization vessel have been copolymerized.

If first a monomer is polymerized and then the second monomer is added, it is possible to obtain graft copolymers which have a core/shell structure, as revealed in Adolf Echte, Handbuch der technischen Polymerchemie, VCH Verlag, Weinheim, 1993, pp. 339 ff. In this case it is the hydrophilicity of the monomers used that determines which monomer is incorporated in the interior and which in the exterior of the graft copolymer. Depending on the parameters of the experiment, it is possible to establish other morphologies as well, which are described in more detail in S. Kirsch, K. Landfester, O. Shaffer, M. S. El-Aasser, Acta Polym. 1999, 50, 347-362. The processes for preparing graft copolymers may also be conducted in succession with more than two monomers.

Reaction Temperature

The reaction temperature depends on the chemical nature of the initiator system, so that the entire range from 0 to 100° C. when operating under atmospheric pressure or under subatmospheric pressure may be considered. Preference is given, however, to temperatures of from 70 to 100° C., with particular preference from 80 to 100° C., and with very particular preference from 85 to 100° C. When working under superatmospheric pressure, however, the reaction temperature may exceed 100° C. and may be up to 230° C. or more.

Reaction Pressure

The reactions may be conducted under atmospheric, superatmospheric or subatmospheric pressure. In general, the reactions are conducted at from 0.01 to 1 000 bar, preferably from 0.01 to 200 bar, with particular preference from 0.01 to 50 bar with very particular preference from 0.01 to 15 bar. Highly volatile monomers such as ethylene, butadiene or vinyl chloride are preferably polymerized under superatmospheric pressure. The copolymerization of styrene and butadiene takes place preferably at from 0.01 to 15 bar.

Mode of Mixing

Depending on the nature of the reaction sites, the mixing of the reaction solution may take place in a variety of ways. Conventionally, mixing takes place by stirring with magnetic stirrers.

In the synthesis system already described above, the mixing of the reaction medium may take place, for example, by means of a horizontal circular shaking movement of the reaction vessels, known as vortexing. Better mixing may additionally be ensured by means of fixed glass rods which are installed in the vortexing reaction vessels and which act as flow breakers.

Experimental Series

In an experimental series, n reaction mixtures are conducted in parallel at n spatially separate reaction sites. Each of the n reaction mixtures differs in at least one reaction parameter in each case from every other reaction mixture. This makes it possible to obtain information on structure/property relationships of polymers and on the influence of reaction parameters on the resulting n aqueous polymer dispersions and to do so within a very short time. The parameters which may be varied include the nature and amount of the monomers, dispersants, initiators and further suitable components used, the amount of the reaction medium, the reaction temperature, the reaction pressure, the mode of mixing, the nature of the conduct of the polymerization reaction, and other parameters such as the simultaneous linear, simultaneous nonlinear, successive linear or successive nonlinear feed of components, which may have an influence on the properties of the polymer dispersions obtained.

If, for example, the amount of a monomer is varied while all other parameters are left constant, it is possible to investigate how the properties of the n aqueous polymer dispersions formed change as a function of the amount of the incorporated monomer. Another possible approach is to vary the nature of one of the monomers used. For example, one or two monomers may be left constant while one representative in each case of a homologous series is used as the third monomer. It is also possible to replace one monomer successively by another.

In one embodiment of the invention, for example, the composition of the first reaction mixture is 10% monomer A and 90% monomer B. Then, in the following reaction mixtures, monomer B is replaced successively by monomer A, so that in a 35 second reaction mixture there is 20% monomer A and 80% monomer B, and in a third reaction mixture there is 30% monomer and 70% monomer B, etc.

This produces a very large number of variation possibilities, since it is also possible to change two or more parameters simultaneously. For example, the amount of a monomer may be varied in ten reaction mixtures. In the second ten reaction mixtures, then, this monomer may be replaced by another, but the amount thereof varied in the same way, etc. In one experimental series, therefore, both the nature and the amount of a monomer have been varied.

Similarly, it is also possible to vary the other reactants such as dispersants, initiators and further suitable components. It is also possible to vary the concentrations of the reactants used by way of the amount of the reaction medium.

Further possibilities of the combinatorial technique are the variation of the reaction temperature, of the reaction pressure, of the mode of mixing, the way in which the polymerization reaction is conducted, and the way in which the components are supplied. The emulsion polymerization may be conducted both in one stage and in two or more stages, continuously, semicontinuously and batchwise. Here again it is possible and rational to alter two or more parameters simultaneously within an experimental series. For example, the reaction may be conducted continuously in one stage in 10 reaction batches, batchwise in one stage in a further 10, multistage and continuously in a further 10, etc.

Formulations of Aqueous Polymer Dispersions

The invention likewise embraces processes for preparing an array of m formulations of polymer dispersions in m parallel mixtures, comprising the steps of

(a) providing in each case one aqueous polymer dispersion to m spatially separate sites for the m mixtures,

(b) providing in each case one further aqueous polymer dispersion and/or a further suitable component to each of the m spatially separate sites,

(c) mixing the aqueous polymer dispersions (a) with the further aqueous polymer dispersions (b) and/or the further components (b) at the m spatially separate sites for the m mixtures, each of the m mixtures differing from every other reaction mixture in at least one parameter in each case, selected from the group consisting of nature and amount of the aqueous polymer dispersions used, composition of the aqueous polymer dispersions used, nature and amount of the further suitable components used, and further parameters which may have an influence on the properties of the resulting formulations of polymer dispersions,

(d) parallel and/or serial characterization of the resulting m formulations of polymer dispersions for at least one property that is of interest,

m being an integer greater than or equal to 1.

Formulations are mixtures of aqueous polymer dispersions with other polymer dispersions and/or with further appropriate components. The nature, number and amount of the aqueous polymer dispersions and further appropriate components used is dependent on the desired composition of the formulation. In general, the further appropriate components such as mineral and organic substances are added in solid form.

As aqueous polymer dispersions in the process of the invention it is possible to use the aqueous polymer dispersions preparable by the emulsion polymerization processes described above. In one preferred embodiment, an aqueous polymer dispersion prepared by the abovementioned emulsion polymerization processes on an industrial scale and optimized in terms of its properties is used as standard polymer dispersion for the formulation mixtures. It is then distributed over the m spatially separate locations and is mixed, for example, with other aqueous polymer dispersions. As other aqueous polymer dispersions it is likewise possible to use those preparable by above-described emulsion polymerization processes. In general, two or more aqueous polymer dispersions which differ in terms of their composition and/or their properties will be mixed with one another. For example, an aqueous polymer dispersion which may be used as a pressure sensitive adhesive is mixed with another aqueous polymer dispersion which has a higher strength, in order thus to give a mixture having optimized properties.

The mineral and organic substances which may be mixed with the aqueous polymer dispersions include, for example, dyes and fillers.

In order to obtain formulations having optimized properties rapidly, the experimental series will be configured in analogy to the experimental series in the case of the emulsion polymerization, i.e., each of the m spatially separate mixtures will differ from the other reaction mixtures in respect of a parameter which may have an influence on the properties of the formulations obtained. Such parameters include, for example, the nature and amount of the aqueous polymer dispersions used, the composition of the aqueous polymer dispersions used, the nature and amount of the further components used, and the mixing proportions of the individual constituents of the formulations with respect to one another. The temperature at which mixing takes place may also affect the stability and the inherent nature of the formulation.

To produce an array, there are numerous variants depending on the parameter altered. A common feature of these variants is that, in an experimental series for the production of an array, m mixtures are conducted in parallel at m spatially different reaction locations. The synthesis systems discussed already in the context of the emulsion polymerization processes are employed here. The rapid production of a very wide variety of arrays makes it possible, within a very short time, to optimize properties such as adhesion, strength and coloring power of aqueous polymer dispersions.

If, for example, the amount of a dye is varied while the amount of the aqueous polymer dispersion is kept constant, it is possible to investigate how the properties of the m resultant formulations change as a function of the amount of the dye. Another possible approach is to vary the nature of one of the aqueous polymer dispersions used. For example, the amount of dye may be kept constant but mixed in each case with an aqueous polymer dispersion of a different polymer. It is also possible to mix two aqueous polymer dispersions with one another while successively replacing one aqueous polymer dispersion by the other.

In one embodiment of the invention, for example, in the first mixture a composition is 10% of aqueous polymer dispersion A and 90% of aqueous polymer dispersion B. In the following reaction mixtures, the aqueous polymer dispersion B is then successively replaced by the aqueous polymer dispersion A, so that in a second reaction mixture there is 20% of aqueous polymer dispersion A and 80% of aqueous polymer dispersion B and in a third reaction mixture there is 30% of aqueous polymer dispersion A and 70% of aqueous polymer dispersion B, and so on.

It is also possible to mix two aqueous polymer dispersions with a dye, keeping the amount of dye constant while successively replacing one aqueous polymer dispersion by the other. Instead of a dye or additionally, it is also possible to add other components as well, such as fillers.

The result is a very large number of variation possibilities, since two or more parameters may also be altered simultaneously. For example, a constant amount of aqueous polymer dispersion A may be introduced in all m mixtures. In the first ten mixtures, the amount of another aqueous polymer dispersion B is then varied. In the second ten mixtures, this aqueous polymer dispersion B may then be replaced by the aqueous polymer dispersion C, which amount is, however, varied in the same way, etc. In one experimental series, accordingly, both the nature and the amount of an aqueous polymer dispersion have been varied.

In general, the aqueous polymer dispersions or the further appropriate components such as dyes and fillers are added batchwise.

Locations used for mixing the aqueous polymer dispersions with the other aqueous polymer dispersions and/or further appropriate components may be the same locations which acted as reaction locations for the above-described emulsion polymerization processes.

Depending on the nature of the locations, the mixing of the formulations may take place in a variety of ways. Conventionally, mixing takes place by stirring using magnetic stirrers.

In the synthesis system already described above, for example, the mixing of the formulations may take place by means of a horizontal circular shaking movement of the reaction vessels, known as vortexing. Better mixing may additionally be ensured by means of permanently installed glass rods which are accommodated in the vortexing reaction vessels and act as flow disruptors.

Parallel and/or Serial Characterization in Very General Terms

The n aqueous polymer dispersions and m formulations of aqueous polymer dispersions preparable by the processes of the invention may be characterized by techniques known to the skilled worker, as described in D. Distler (editor), WaBrige Polymerdispersionen: Synthese, Eigenschaften, Anwendungen, Wiley-VCH-Verlag Weinheim, 1999, pp. 31-65.

### EXAMPLES

The following description of reaction mixtures for emulsion polymerization processes is exemplary in nature. In this case, variations have been made in the monomer composition, the size of the mixtures, the mixing of the reaction solution, and the addition of the reactants, which took place either batchwise or continuously. This made it possible to investigate the effect of these reaction parameters on the particle size and, if appropriate, its distribution.

Table 1 reproduces the formulas for Examples 1 and 3 to 5. The dispersant used was the emulsifier Emulphor NPS® from BASF AG. Mixtures R1 to R16 contained the monomer mixtures specified below and also 3706 mg of water and 194 mg of a 31% strength by weight aqueous solution of emulsifier.

TABLE 1

| Reactor | |
|---|---|
| No. | Initial monomer charge |
| R1 | 1980 mg 2-ethylhexyl acrylate, 20 mg acrylic acid |
| R2 | 1960 mg 2-ethylhexyl acrylate, 40 mg acrylic acid |
| R3 | 1940 mg 2-ethylhexyl acrylate, 60 mg acrylic acid |
| R4 | 1920 mg 2-ethylhexyl acrylate, 80 mg acrylic acid |
| R5 | 1980 mg n-butyl acrylate, 20 mg acrylic acid |
| R6 | 1960 mg n-butyl acrylate, 40 mg acrylic acid |
| R7 | 1940 mg n-butyl acrylate, 60 mg acrylic acid |
| R8 | 1920 mg n-butyl acrylate, 80 mg acrylic acid |
| R9 | 900 mg n-butyl acrylate, 20 mg acrylic acid, 990 mg styrene |
| R10 | 980 mg n-butyl acrylate, 40 mg acrylic acid, 980 mg styrene |
| R11 | 970 mg n-butyl acrylate, 60 mg acrylic acid, 970 mg styrene |
| R12 | 960 mg n-butyl acrylate, 80 mg acrylic acid, 960 mg styrene |
| R13 | 990 mg n-butyl acrylate, 20 mg acrylic acid, 990 mg methyl |
| | methacrylate |
| R14 | 980 mg n-butyl acrylate, 40 mg acrylic acid, 980 mg methyl |
| | methacrylate |
| R15 | 970 mg n-butyl acrylate, 60 mg acrylic acid, 970 mg methyl |
| | methacrylate |
| R16 | 960 mg n-butyl acrylate, 80 mg acrylic acid, 960 mg methyl |
| | methacrylate |

### Example 1

### Batchwise Reaction Procedure:

Water and emulsifier were charged to the reactors. The respective quantities of monomer were mixed and then pipetted in simultaneously, and were emulsified by shaking in the reactors. The emulsions were heated to 90° C. Over a period of 120 minutes, 33.3 μl of the initiator solution, consisting of 10 mg of sodium persulfate in 990 mg of water, were metered in every 4 minutes. Subsequently, the mixtures were cooled to room temperature.

### Example 2

### Batchwise Reaction Procedure in a Laboratory Reactor

The reactions were conducted in a 4 l glass laboratory reactor, prior art to the skilled worker, with a plurality of feeds. Table 2 reproduces the amount of monomers used.

TABLE 2

| D1 | D2 | D3 | D4 |
|---|---|---|---|
| Initial charge: | Initial charge: | Initial charge: | Initial charge: |
| 396 g | 384 g n-butyl | 196 g n-butyl | 194 g n-butyl |
| ethylhexyl | acrylate | acrylate | acrylate |
| acrylate | 16 g acrylic acid | 196 g styrene | 194 g methyl |
| 4 g acrylic acid | | 8 g acrylic acid | methacrylate |
| | | | 12 g acrylic acid |

The monomer mixtures specified in Table 2 were emulsified in a mixture of 741 g of water and 39 g of a 31% by weight aqueous solution of emulsifier, and heated to 90° C. The initiator solution (196 g of water +2 g of sodium persulfate) was added by the feed technique over the course of 2 hours. Subsequently, polymerization was continued for 30 minutes, after which the mixtures were cooled to room temperature.

The results of Examples 1 and 2 are given in Table 3. d₅₀ is the centrifuge average of the particle diameter. The dispersions were characterized by their particle size distribution as determined using the analytical ultracentrifuge (AUC). Regarding the description of this technique, and sample preparation, reference should be made to W. Machtle, S. Harding (ed.), AUC in Biochemistry and Polymer Science, Cambridge, UK, 1991, pp. 147-175 and W. Machtle, Angew. Makromol. Chem. 1988, Vol. 162, p. 35.

TABLE 3

| No. | R1 | R2 | R3 | R4 | R5 | R6 | R7 | R8 | R9 | R10 |
|---|---|---|---|---|---|---|---|---|---|---|
| d₅₀ | 67 | 61 | 60 | 58 | 55 | 53 | 59 | 52 | 50 | 45 |
| [nm] | | | | | | | | | | |
| No. | R11 | R12 | R13 | R14 | R15 | R16 | D1 | D2 | D3 | D4 |
| d₅₀ | 42 | 45 | 49 | 52 | 48 | 57 | — | — | 44 | 45 |
| [nm] | | | | | | | | | | |

In the case of batchwise reaction procedure, a narrow particle size distribution was found whose maximum is virtually independent of the monomer composition. No significant difference is found between the particle size distributions of the experiments in the parallel reactor on the 7 ml scale and the laboratory experiments in the 4 l reactor. All the samples have a small average particle diameter. Accordingly, the experimental system used is outstandingly suitable for conducting model reactions on a small, cost-effective scale, which can then be scaled up without problems.

### Example 3

### Semicontinuous Reaction Procedure

In a reactor block with 16 reactors, monomer, initiator and emulsifier were in each case emulsified in water. 25% by weight of these mixtures in each case was then removed, introduced as initial charge in a second reactor block with 16 reactors, and heated to 90° C. The remaining 75% was then added to this initial charge by pipette over a period of 120 minutes.

### Example 4

### Semicontinuous Reaction Procedure Using Glass Rods as Flow Disruptors in the Reactor Blocks

The procedure was as in Example 3 but using glass rods as flow disruptors.

### Example 5

### Semicontinuous Reaction Procedure with Seed Addition

The procedure was as in Example 3 but with a deviation in the formula, as evident from Table 4, involving the addition of 0.1% by weight of linear polystyrene beads as seed particles to the initial charge. Four dispersions from each of the three experiments were again analyzed for their particle size distribution by means of the analytical ultracentrifuge, as shown in Table 4. Narrow monomodal particle size distributions are obtained with a larger average particle diameter d₅₀% than in the case of the batchwise reaction procedure, in which particles having a size of from 81 to 107 nm are obtained. The particle size distribution may be expressed by the factor (d_{90%}-d_{10%})/d_{50%}. The smaller this factor, the narrower the particle size distribution.

TABLE 4

| Mixing ratio of the | Reaction | d_{50%} | |
|---|---|---|---|
| monomers | procedure | [nm] | (d_{90%}-d_{10%})/d_{50%} |
| 99 2-ethylhexyl acrylate | Feed | 92 | 0.09 |
| 1 acrylic acid | Feed with flow | 85 | 0.09 |
| | disruptors | | |
| | Feed with initial | 88 | 0.17 |
| | charge of fine seed | | |
| 96 n-butyl acrylate | Feed | 106 | 0.16 |
| 4 acrylic acid | Feed with flow | 86 | 0.09 |
| | disruptors | | |
| | Feed with initial | 97 | 0.17 |
| | charge of fine seed | | |
| 49 n-butyl acrylate | Feed | 106 | 0.22 |
| 49 methyl methacrylate | Feed with flow | 88 | 0.28 |
| 2 acrylic acid | disruptors | | |
| | Feed with initial | 95 | 0.20 |
| | charge of fine seed | | |
| 48.5 n-butyl acrylate | Feed | 89 | 0.07 |
| 48.5 styrene | Feed with flow | 86 | 0.11 |
| 3 acrylic acid | disruptors | | |
| | Feed with initial | 86 | 0.10 |
| | charge of fine seed | | |

We claim:

1. A process for preparing an array of n polymer dispersions by emulsion polymerization in n parallel reaction mixtures, comprising the steps of
(a) providing two or more monomers, one or more initiators, one or more dispersants, water as reaction medium and, if desired, further useful components suitable for emulsion polymerization at n spatially separate reaction sites for the n reaction mixtures,
(b) causing the simultaneous or successive reaction of the two or more monomers and, if appropriate, of the further useful components, each of the n reaction mixtures differing from any other reaction mixture in at least one reaction parameter in each case, selected from the group consisting of nature and amount of the monomers, dispersants, initiators and further suitable components used, amount of the reaction medium, reaction temperature, reaction pressure, type of mixing, mode of conduct of the polymerization reaction, and other parameters which may have an influence on the properties of the resulting polymer dispersions,
(c) carrying out parallel and/or serial characterization of the resulting n polymer dispersions for at least one property which is of interest,
n being an integer greater than or equal to 1.

2. A process as claimed in claim 1, wherein at least two different monomers containing at least one ethylenically unsaturated group are reacted.

3. A process as claimed in either of claims 1 and 2, wherein at least one hydrophilic monomer and one hydrophobic monomer are reacted with one another.

4. A process as claimed in any of claims 1 to 3, wherein the spatially separate reaction locations are wells in a microtitre plate or glass vessels of a parallelized synthesis system.

5. A process as claimed in any of claims 1 to 4, wherein further useful components added comprise seed particles and/or dyes and/or other components which are not copolymerized.

6. A process as claimed in any of claims 1 to 5, wherein the emulsion polymerization is a miniemulsion polymerization.

7. A process as claimed in any of claims 1 to 6, wherein the reaction mixtures are mixed by means of horizontal circular shaking motion of reaction vessels.

8. A process as claimed in claim 7, wherein glass rods are installed as flow disruptors in the reaction vessels.

9. A process as claimed in any of claims 1 to 8, wherein n is an integer greater than 10.

10. A process as claimed in any of claims 1 to 8, wherein n is an integer greater than 100.

11. A process as claimed in any of claims 1 to 8, wherein n is an integer greater than 1 000.

12. An array of n aqueous polymer dispersions preparable by a process as claimed in any of claims 1 to 11.

13. A process for preparing an array of m formulations of polymer dispersions in m parallel mixtures, comprising the steps of
(a) providing in each case one aqueous polymer dispersion to m spatially separate sites for the m mixtures,
(b) providing in each case one further aqueous polymer dispersion and/or a further suitable component to each of the m spatially separate sites,
(c) mixing the aqueous polymer dispersions (a) with the further aqueous polymer dispersions (b) and/or the further components (b) at the m spatially separate sites for the m mixtures, each of the m mixtures differing from every other reaction mixture in at least one parameter in each case, selected from the group consisting of nature and amount of the aqueous polymer dispersions used, composition of the aqueous polymer dispersions used, nature and amount of the further suitable components used, and further parameters which may have an influence on the properties of the resulting formulations of polymer dispersions,
(d) parallel and/or serial characterization of the resulting m formulations of polymer dispersions for at least one property that is of interest,
m being an integer greater than or equal to 1.

14. A process as claimed in claim 13, wherein n is an integer greater than 10.

15. A process as claimed in claim 13, wherein n is an integer greater than 100.

16. A process as claimed in claim 13, wherein n is an integer greater than 1 000.

17. An array of m formulations of aqueous polymer dispersions preparable by a process as claimed in any of claims 13 to 16.
